(19) United States
(12) Patent Application Publication
(10) Pub. No.: US 2007/0088332 A1
(43) Pub. Date: Apr. 19, 2007

(54) Iontophoresis device

(75) Inventors: Hidero Akiyama, Shibuya-ku (JP); Mizuo Nakayama, Shibuya-ku (JP); Takehiko Matsumura, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/508,732

(22) Filed: Aug. 22, 2006

Related U.S. Application Data
(60) Provisional application No. 60/719,343, filed on Sep. 20, 2005.

(30) Foreign Application Priority Data
Aug. 22, 2005 (JP) 2005-240460

(51) Int. Cl. A61K 9/22

(52) U.S. Cl. 604/890.1

(57) ABSTRACT

Contamination between an active agent solution in an active agent reservoir and an electrolyte solution in an electrolyte solution reservoir may be reduced in an iontophoresis device, thus helping to suppress the generation of gas and helping to reduce changes in pH upon energization. A gel matrix that transforms into a liquid state upon thermal excitation and/or mechanical excitation may be used in one or more reservoirs in the iontophoresis device.

### CROSS-REFERENCES TO RELATED APPLICATIONS

This application claims the benefit to Japanese Patent Application No. 2005-240460 and also claims benefit under 35 U.S.C. § 119(e) of U.S. Provisional Patent Application No. 60/719,343, filed Sep. 20, 2005, both of which are incorporated herein by reference in their entireties.

### BACKGROUND

1. Field

The present disclosure relates to an iontophoresis device for administering active agent ions to a subject.

2. Description

Iontophoresis is a method of delivering an active agent into a subject through a biological membrane of the subject. An iontophoresis device may include an active electrode assembly comprising an active agent reservoir holding an active agent solution, and a counter electrode assembly as a counter electrode to the active electrode assembly. An electric potential having the same polarity as that of active agent ions in the active agent reservoir may be applied to the active electrode assembly with the active agent solution contacting the biological membrane to electrically drive and transfer the active agent ions into the subject via the biological membrane.

WO 03/037425 A1 discloses an iontophoresis device that comprising an active electrode assembly and a counter electrode assembly, where each assembly is constructed using membranes. Dissimilar ion exchange membranes are provided to the active electrode assembly. One ion exchange membrane selectively passes ions having the same charge as active agent ions, while the other ion exchange membrane selectively passes ions opposite in polarity to the active agent ions. In addition, at least one ion exchange membrane is provided to the counter electrode assembly. The at least one ion exchange membrane selectively passes ions opposite in polarity to the active agent ions. The iontophoresis device disclosed in WO 03/037425 A1 may be capable of administering an ionic active agent stably, and with a high transport efficiency, over a long time period.

An iontophoresis device may be constructed by using a gel matrix as an active agent reservoir, which holds an ionic active agent, or as an electrolyte solution reservoir, which holds an electrolyte solution. One potential problem with using a gel matrix as an active agent reservoir or an electrolyte solution reservoir is that gas may be generated during use of the device at points where the gel matrix comes into contact with an electrode. Using a liquid instead of a gel to configure the active agent reservoir and/or the electrolyte reservoir may lead to a different problem, that is potential contamination between the active agent reservoir and the electrolyte solution reservoir before the device is used.

### BRIEF SUMMARY

In one aspect, the present disclosure is directed to an iontophoresis device comprising an active agent reservoir and an electrolyte solution reservoir, each reservoir comprising a gel matrix used to reduce contamination. Each gel matrix is adapted to reduce gas generation at contact points between the gel matrix and an electrode, thus reducing pH changes in an active agent solution and/or an electrolyte solution.

In one aspect, the present disclosure is directed to an iontophoresis device comprising an active electrode assembly, a counter electrode assembly, and a DC electric power source. The active electrode assembly may comprise an active electrode; an electrolyte solution reservoir holding an electrolyte solution, the electrolyte solution reservoir placed on an outer surface of the active electrode; a second ion exchange membrane that selectively passes ions opposite in polarity to the active agent ions, the second ion exchange membrane placed on an outer surface of the electrolyte solution reservoir; an active agent reservoir holding the ionic active agent, the active agent reservoir placed on an outer surface of the second ion exchange membrane; and a first ion exchange membrane that selectively passes ions having the same polarity as that of the active agent ions, the first ion exchange membrane placed on an outer surface of the active agent reservoir. The DC electric power source may be connected to the active electrode. The electrolyte solution reservoir and/or the active agent reservoir may comprise a gel matrix that transforms into a liquid upon thermal or mechanical excitation.

The counter electrode assembly may comprise: a counter electrode; a second electrolyte solution reservoir that holds a second electrolyte solution, the second electrolyte solution reservoir placed on an outer surface of the counter electrode; a third ion exchange membrane that selectively passes ions having the same polarity as the active agent ions, the third ion exchange membrane placed on an outer surface of the second electrolyte solution reservoir; a third electrolyte solution reservoir holding a third electrolyte solution, the third electrolyte solution reservoir placed on an outer surface of the third ion exchange membrane; and a fourth ion exchange membrane that selects ions having a polarity opposite to that of the active agent ions, the fourth ion exchange membrane placed on the front surface of the third electrolyte solution reservoir. The DC electric power source may be connected to the counter electrode. The second electrolyte solution reservoir and/or the third electrolyte solution reservoir may comprise a gel that transforms into a liquid upon thermal or mechanical excitation.

Using a gel in the active agent reservoir and/or the electrolyte solution reservoir may help reduce contamination of the active agent solution and/or the electrolyte solution during storage or the iontophoresis device. Transforming the gel into a liquid state by thermal or mechanical excitation during use of the device may help to suppress the generation of gas at points of contact between with the active and/or counter electrodes, as well as resulting changes in pH.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a top plan view showing an iontophoresis device.

FIG. 2 is an enlarged sectional view taken along the line II-II of FIG. 1.

FIG. 3 is an enlarged sectional view taken along the line III-III of FIG. 1.

FIG. 4 is a sectional view showing a main portion of an iontophoresis device.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, electric potential or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this-specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating iontophoretic active agent delivery including “a controller” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

A used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

A used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., an active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizeable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the -caine class; morphine, hydromorphone, fentanyl, oxycodone, hydrocodone, buprenorphine, methadone, and similar opiod agonists; sumatriptan succinate, zolmitriptan, naratriptan HCl, rizatriptan benzoate, almotriptan malate, frovatriptan succinate and other 5-hydroxytryptaminel receptor subtype agonists; resiquimod, imiquidmod, and similar TLR 7 and 8 agonists and antagonists; domperidone, granisetron hydrochloride, ondansetron and such anti-emetic active agents; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes active agents such as exenatide; as well as peptides and proteins for treatment of obesity and other maladies.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

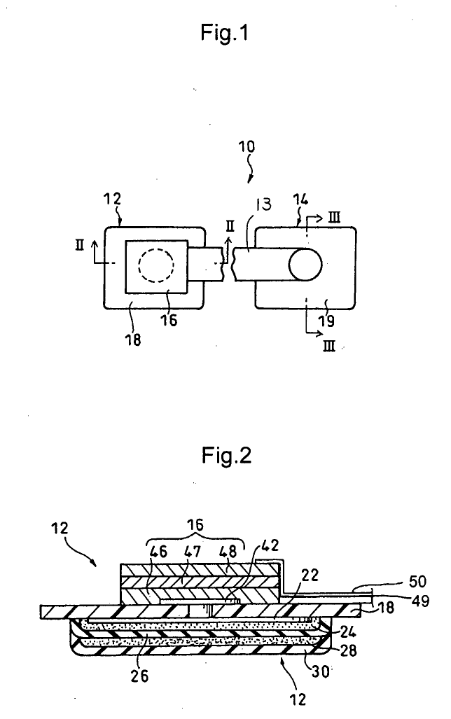

Referring to FIGS. 1 to 3, an iontophoresis device 10 comprises an active electrode assembly 12, a counter electrode assembly 14, and a DC electric power source 16. The electrode assemblies 12 and 14 are connected to opposite polarity terminals of the DC electric power source 16.

The active electrode assembly 12 may comprise an active electrode 22, an electrolyte solution reservoir 24, a second ion exchange membrane 26, an active agent reservoir 28, and a first ion exchange membrane 30 in order from a base sheet 18.

The active electrode 22 may comprise a conductive coating applied to an outer surface of the base sheet 18, blended with a non-metallic conductive filler such as a carbon paste. A copper plate or a metallic thin film may also be used for the active electrode 22. However, it may be advantageous to use the conductive coating as the active electrode 22 in order to prevent metal from the plate or thin film from eluting and possibly transferring to a subject upon administration of an active agent.

The electrolyte solution reservoir 24 may comprise a gel matrix placed in contact with the active electrode 22. An electrolyte that oxidizes or reduces more easily than an electrolytic reaction of water (oxidation at a positive electrode and reduction at a negative electrode) occurs may be advantageous. Examples of such electrolytes include: medical agents such as ascorbic acid (vitamin C) and sodium ascorbate; and organic acids such as lactic acid, oxalic acid, malic acid, succinic acid, and fumaric acid and/or salts thereof. The use of such electrolytes may suppress the generation of an oxygen and/or hydrogen gas. In addition, blending a plurality of electrolytes to form a buffer may help to suppress changes in pH when the iontophoresis device is energized.

The second ion exchange membrane 26 may comprise an ion exchange resin, into which an ion exchange group is introduced as a counter ion. The counter ion has a polarity opposite to that of active agent ions in the active agent reservoir 28. An anion exchange resin may be used in the second ion exchange membrane 26 when the active agent ions in the active agent reservoir 28 are cations. A cation exchange resin may be used in the second ion exchange membrane 26 when the active agent ions in the active agent reservoir 28 are anions.

The active agent reservoir 28 is obtained by causing an active agent (or a precursor for the active agent) dissolved in a solvent, where the active agent dissociates into positive or negative active agent ions, to gel. Examples of an active agent whose active agent component dissociates to positive ions include the anesthetic active agents lidocaine hydrochloride and morphine hydrochloride. Examples of an active agent whose active agent component dissociates into negative ions include the vitamin agent ascorbic acid.

The first ion exchange membrane 30 may comprise an ion exchange resin, into which an ion exchange group is introduced as a counter ion. The counter ion has the same polarity as that of the active agent ions in the active agent reservoir 28. A cation exchange resin may be used in the first ion exchange membrane 30 when the active agent ions in the active agent reservoir 28 are cations. An anion exchange resin may be used in the first ion exchange membrane 30 when the active agent ions in the active agent reservoir 28 are anions.

Without limitation, cation exchange resins may be obtained by introducing a cation exchange group (an exchange group using a cation as a counter ion) such as a sulfonic group, a carboxylic group, or a phosphoric group into a polymer having a three dimensional network structure, such as a hydrocarbon based resin (for example, a polystyrene resin or an acrylic resin) or a fluorine based resin having a perfluorocarbon skeleton.

Without limitation, anion exchange resins may be obtained by introducing an anion exchange group (an exchange group using an anion as a counter ion) such as a primary amino group, a secondary amino group, a tertiary amino group, a quaternary ammonium group, a pyridyl group, an imidazole group, a quaternary pyridinium group, or a quaternary imidazolium group into a polymer having a three dimensional network structure such as a hydrocarbon based resin (for example, a polystyrene resin or an acrylic resin) or a fluorine based resin having a perfluorocarbon skeleton.

The gel matrix that comprises the electrolyte solution reservoir 24 and/or the active agent reservoir 28 may advantageously take the form of a gel that changes to a liquid upon thermal excitation, such as a gelatinous gel or a starch-like gel.

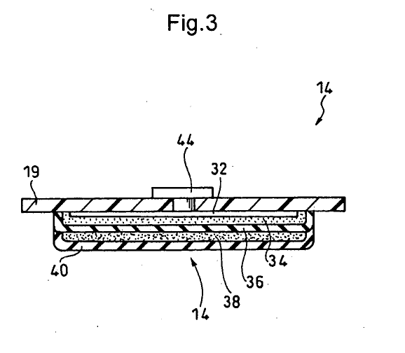

FIG. 3 is a partial enlarged view showing that the counter electrode assembly 14 may comprise a counter electrode 32, a second electrolyte solution reservoir 34, a third ion exchange membrane 36, a third electrolyte solution reservoir 38, and a fourth ion exchange membrane 40 in order from a base sheet 19 similar to the base sheet 18.

The counter electrode 32 may be similar to the active electrode 22 in the active electrode assembly 12. Further, the second electrolyte solution reservoir 34 and the third electrolyte solution reservoir 38 may comprise gels similar to that used in the electrolyte solution reservoir 24.

The third ion exchange membrane 36 may comprise an ion exchange resin similar to that used in the first ion exchange membrane 30, and may thus function similarly to the first ion exchange membrane 30.

The fourth ion exchange membrane 40 may comprise an ion exchange resin similar to that used in the second ion exchange membrane 26, and may thus function similarly to the second ion exchange membrane 26.

An active electrode terminal 42 may be arranged on another other surface of the base sheet 18, and a connection may be established between the active electrode terminal 42 and the active electrode 22 of the active electrode assembly 12 via a through-hole formed in the base sheet 18.

Similarly, a counter electrode terminal 44 may be arranged on another surface of the base sheet 19, and a connection may be established between the counter electrode terminal 44 and the counter electrode 32 of the counter electrode assembly 14 via a through-hole formed on the base sheet 19.

The DC electric power source 16 may be placed between the active electrode terminal 42 and the counter electrode terminal 44. The DC electric power source 16 may comprise a cell type battery that includes a first active electrode layer 46, a separator layer 47, and a second active electrode layer 48 laminated sequentially on one surface of the base sheet 18 by using a method such as printing. The first active electrode layer 46 of the DC electric power source 16 and the active electrode terminal 42 may be directly coupled together. The second active electrode layer 48 and the counter electrode terminal 44 may be coupled together by using a coating film (conductive layer) 45 of a conductive paint or ink formed on an insulating paste layer 49.

Reference numeral 13 in FIG. 1 denotes a coupling belt that may be used to couple the active electrode assembly 12 and the counter electrode assembly 14. The coating film 45 may also be applied to the coupling belt 13, and may extend up to the counter electrode terminal 44.

The structure of the DC electric power source 16 is not limited to the embodiment described here. Thin cell batteries disclosed in JP 11-067236 A, US 2004/0185667 A1, and U.S. Pat. No. 6,855,441 may also be used for the DC electric power source 16.

The active electrode assembly 12 and the counter electrode assembly 14 may be heated by being brought into contact with the biological membrane of a subject when the iontophoresis device 10 is to be used. The electrode assemblies may thus readily heat up to a temperature near the body temperature of the subject using the iontophoresis device.

Heating causes the gels comprising the active agent reservoir 28, the electrolyte solution reservoir 24, the second electrolyte solution reservoir 34, and the third electrolyte solution reservoir 38 to transform into liquid active agent solutions and electrolyte solutions. As a result, a liquid is present at the contact surface between the gel and the active electrode 22 and at the surface of contact between the gel and the counter electrode 32. The generation of gas may thus be reduced, and changes in pH may thus be suppressed.

The active agent solution and the electrolyte solution are stored in a gel state, thus reducing contamination between the electrolyte solution and the active agent component in the active agent reservoir 28.

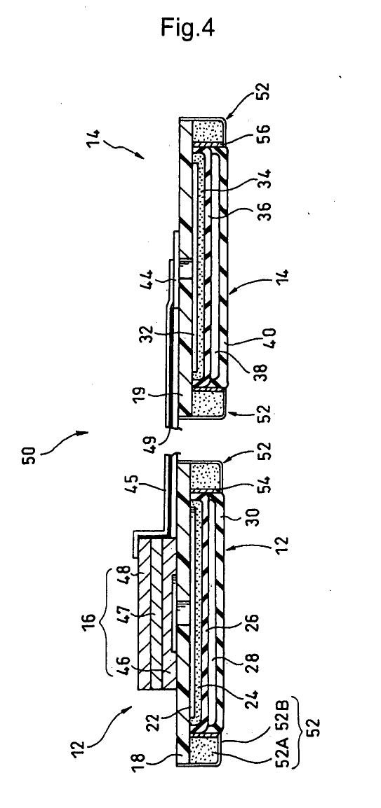

FIG. 4 shows another embodiment of an iontophoresis device.

An iontophoresis device 50 comprises a liquefying device 52 for liquefying the active agent reservoir 28, the electrolyte solution reservoir 24, the second electrolyte solution reservoir 34, and the third electrolyte solution reservoir 38. In addition, the active electrode assembly 12 and the counter electrode assembly 14 may be housed in container shape cells 54 and 56, respectively, with ion exchange membranes at distal ends exposed. Other constituent features are similar to those of the iontophoresis device 10.

The liquefying device 52 may comprise a heating device if the gel matrix transforms to a liquid upon thermal excitation. Alternatively, the liquefying device 52 may comprise a mechanical excitation device such as an ultrasonic generator if the gel transforms to a liquid upon mechanical excitation. When a heating device is employed, the liquefying device 52 may comprise an iron oxidation exothermic material 52A and a seal 52B that hermetically seals the material out of contact with the air. The iron oxidation exothermic material 52A is placed outside the cells 54 and 56, and the outside of the material is covered with the seal 52B.

By removing the seal 52B when the iontophoresis device 50 is used, the iron oxidation exothermic material 52A is brought into contact with oxygen in the atmosphere, causing the material to oxidize. Heat of combustion may heat the electrolyte solution reservoir 24 and the second electrolyte solution reservoir 34 to a temperature sufficient to transform the gel matrices therein to liquids. As a result, a liquid is present at the contact surface between the gel and the active electrode 22 and at the surface of contact between the gel and the counter electrode 32. The generation of gas may thus be reduced, and changes in pH may thus be suppressed.

Furthermore, the liquefying device 52 also heats the biological membrane of a subject, which tends to enhance permeation of an active agent solution into the subject.

The liquefying device 52 is not limited to the iron, oxidation exothermic material 52A. In an alternative heating device, a surface exothermic body that generates heat by virtue of energization may be wrapped around the cells 54 and 56. A heating device may also be placed on the outer sides in FIG. 4 of the base sheets 18 and 19 to heat the gel via the electrode terminals 42 and 52.

Examples of gels that transform to a liquid state upon mechanical excitation include gels having an added thixotropy modifier or an added viscosity modifier. The viscosity of the gel may be reduced through mechanical excitation (applying a shear force to) of the gel. An ultrasonic transmitter or a pager (small vibrator) may be used as mechanical exciting devices. The gel matrix should be excited throughout an active agent application period because the liquefied material may revert to a gel state when mechanical excitation is removed. A secondary effect of promoting ionic permeation through the biological interface of the subject may also be present upon application of an ultrasonic wave. Examples of thixotropy modifiers available for use include bentonite, aluminum hydroxide, light anhydrous silicic acid, cross-linkable polyacrylic acid, and cross linkable sodium polyacrylate.

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety.

Aspects of the embodiments can be modified, if necessary, to employ systems, circuits, and concepts of the various patents, applications, and publications to provide yet further embodiments.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. An iontophoresis device used for administering an ionic active agent by iontophoresis comprising:
an active electrode assembly comprising:
an active electrode;
an electrolyte solution reservoir that holds an electrolyte solution, the electrolyte solution reservoir placed on an outer surface of the active electrode;
a second ion exchange membrane that selectively passes ions having a polarity opposite that of the ionic active agent, the second ion exchange membrane placed on an outer surface of the electrolyte solution reservoir;
an active agent reservoir that holds the ionic active agent, the active agent reservoir placed on an outer surface of the second ion exchange membrane; and
a first ion exchange membrane that selectively passes ions having the same polarity as the ionic active agent, the first ion exchange membrane placed on an outer surface of the active agent reservoir,
a counter electrode assembly comprising a counter electrode; and
a DC electric power source connected to the active electrode of the active electrode assembly and to the counter electrode of the counter electrode assembly;
wherein the electrolyte solution reservoir and/or the active agent reservoir comprises a gel matrix that transforms to a liquid upon thermal excitation and/or mechanical excitation.

2. The iontophoresis device according to claim 1, the counter electrode assembly further comprising:
a second electrolyte solution reservoir that holds a second electrolyte solution, the second electrolyte solution reservoir placed on an outer surface of the counter electrode;
a third ion exchange membrane that selectively passes ions having the same polarity as the active agent ions, the third ion exchange membrane placed on an outer surface of the second electrolyte solution reservoir;
a third electrolyte solution reservoir that holds a third electrolyte solution, the third electrolyte solution reservoir placed on an outer surface of the third ion exchange membrane; and
a fourth ion exchange membrane that selectively passes ions having a polarity opposite that of the active agent ions, the fourth ion exchange membrane placed on an outer surface of the third electrolyte solution reservoir;
wherein the second electrolyte solution reservoir and/or the third electrolyte solution reservoir comprises a gel matrix that transforms into a liquid upon thermal excitation and/or mechanical excitation.

3. An iontophoresis device used for administering an ionic active agent by iontophoresis, the iontophoresis device comprising:
an active electrode assembly comprising:
an active electrode;
an active agent reservoir that holds the ionic active agent, the active agent reservoir positionable at least proximate a biological interface of a subject to transdermally deliver the ionic active agent to the biological interface; and
an electrolyte solution reservoir that holds an electrolyte solution, the electrolyte solution reservoir positioned between the active electrode and the active agent reservoir, wherein at least one of the electrolyte solution reservoir or the active agent reservoir comprises a gel matrix that transforms to a liquid upon excitation;
a counter electrode assembly comprising a counter electrode; and
a DC electric power source connected to the active electrode of the active electrode assembly and to the counter electrode of the counter electrode assembly, and operable to apply electrical potentials to the active and the counter electrodes.

4. The iontophoresis device according to claim 3 wherein the electrolyte solution reservoir transforms to a liquid upon thermal excitation.

5. The iontophoresis device according to claim 3 wherein the electrolyte solution reservoir transforms to a liquid upon mechanical excitation.

6. The iontophoresis device according to claim 3 wherein the active agent reservoir transforms to a liquid upon thermal excitation.

7. The iontophoresis device according to claim 3 wherein the active agent reservoir transforms to a liquid upon mechanical excitation.

8. The iontophoresis device according to claim 3, wherein the active electrode assembly further comprises:
an outer ion exchange membrane that selectively passes ions having the same polarity as the ionic active agent, the outer ion exchange membrane placed on an outer surface of the active agent reservoir.

9. The iontophoresis device according to claim 3, wherein the active electrode assembly further comprises:
an inner ion exchange membrane that selectively passes ions having a polarity opposite that of the ionic active agent, the inner ion exchange membrane positioned between the electrolyte solution reservoir and the active agent reservoir.

10. The iontophoresis device according to claim 3 wherein both the electrolyte solution reservoir and the active agent reservoir transforms to a liquid upon excitation.

11. The iontophoresis device according to claim 10, wherein the active electrode assembly further comprises:
an outer ion exchange membrane that selectively passes ions having the same polarity as the ionic active agent, the outer ion exchange membrane positioned between the active agent reservoir and an exterior of the active electrode assembly; and
an inner ion exchange membrane that selectively passes ions having a polarity opposite that of the ionic active agent, the inner ion exchange membrane positioned between the electrolyte solution reservoir and the active agent reservoir.

12. The iontophoresis device according to claim 3 wherein the counter electrode assembly further comprises:
an outer electrolyte solution reservoir that holds an electrolyte solution, the outer electrolyte solution reservoir positionable at least proximate the biological interface of the subject during transdermally deliver of the ionic active agent to the biological interface; and
an inner electrolyte solution reservoir that holds an electrolyte solution, the inner electrolyte solution reservoir placed positioned between the counter electrode and the outer electrolyte solution reservoir, wherein at least one of the inner and the outer electrolyte solution reservoirs comprises a gel matrix that transforms into a liquid upon excitation.

13. The iontophoresis device according to claim 12 wherein the inner electrolyte solution reservoir of the counter electrode assembly transforms into a liquid upon thermal excitation.

14. The iontophoresis device according to claim 12 wherein the inner electrolyte solution reservoir of the counter electrode assembly transforms into a liquid upon mechanical excitation.

15. The iontophoresis device according to claim 12 wherein the outer electrolyte solution reservoir of the counter electrode assembly transforms into a liquid upon thermal excitation.

16. The iontophoresis device according to claim 12 wherein the outer electrolyte solution reservoir of the counter electrode assembly transforms into a liquid upon mechanical excitation.

17. The iontophoresis device according to claim 12 wherein the counter electrode assembly further comprises:
an outer ion exchange membrane that selectively passes ions having a polarity opposite that of the active agent ions, the outer ion exchange membrane positioned between the outer electrolyte solution reservoir and an exterior of the counter electrode assembly.

18. The iontophoresis device according to claim 12 wherein the counter electrode assembly further comprises:
an inner ion exchange membrane that selectively passes ions having the same polarity as the active agent ions, the inner ion exchange membrane positioned between the inner and the outer electrolyte solution reservoirs.

19. The iontophoresis device according to claim 12 wherein both the outer and the inner electrolyte solution reservoirs of the counter electrode assembly transforms into a liquid upon excitation.

20. The iontophoresis device according to claim 19 wherein the counter electrode assembly further comprises:
an outer ion exchange membrane that selectively passes ions having a polarity opposite that of the active agent ions, the outer ion exchange membrane positioned between the outer electrolyte solution reservoir and an exterior of the counter electrode assembly; and
an inner ion exchange membrane that selectively passes ions having the same polarity as the active agent ions, the inner ion exchange membrane positioned between the inner and the outer electrolyte solution reservoirs.
